(19) United States
(12) Patent Application Publication
(10) Pub. No.: US 2014/0107672 A1
(43) Pub. Date: Apr. 17, 2014

(54) Suture Passer and Subcortical Knot Placement

(71) Applicant: Brian D. Dross, Mount Pleasant, SC (US)

(72) Inventor: Brian D. Dross, Mount Pleasant, SC (US)

(73) Assignee: MicroAire Surgical Instruments LLC, Charlottesville, VA (US)

(21) Appl. No.: 14/125,771

(22) PCT Filed: Jun. 18, 2012
(86) PCT No.: PCT/US2012/042949

§ 371 Date: Dec. 12, 2013

Related U.S. Application Data
(60) Provisional application No. 61/499,329, filed on Jun. 21, 2011.

(51) Int. Cl. A61B 17/04

(52) U.S. Cl. CPC A61B 17/0483
USPC 606/144

(57) ABSTRACT

A suture passer (121) and method of using for the exchange of sutures between sections of a device used for attachment of tissue to bone or delivering suture to a separate device with the purpose of pushing then pulling suture through a plane of bone and/or tissue. In one embodiment a U-shaped or teardrop-shaped ridged material (18) (solid wire, braided wire, monofilament extruded polymer) stiffer than the intended suture material is used to pass the suture. Suture material or passing loops may be further contained in a preloaded tube or slotted tube that provides additional stiffening and the elimination of surgical steps. In another embodiment, a method of subcortical-transosseous knot placement is described which increases the load bearing and eliminates post surgical impingement with the acromion.

### CROSS-REFERENCE TO RELATED APPLICATION

The contents of provisional Application U.S. Ser. No. 61/499,329 filed Jun. 21, 2011, on which the present application is based and benefit claimed under 35 U.S.C. §119(e), is herein incorporated by reference.

### BACKGROUND OF THE INVENTION

1. Field of the Invention

This invention relates generally to a timesaving suture passer and its use in arthroscopic transosseous rotator cuff repair to increase repair strength. More particularly, this invention relates to methods and devices for the exchange of suture(s) between sections of a device or delivering suture(s) to a separate device with the purpose of pulling suture(s) through a plane of bone and/or tissue and where benefits of a subcortical knot are realized if used with tunnels of sufficient diameter.

2. Description of the Prior Art

Invasive and open surgery methods of attachment of tissue to bone to repair tissue is well-known. Arthroscopy has become the preferred approach to rotator cuff repair. In performing such surgery, it is common practice to provide a passageway in a bone to reattach a torn or separated tendon to the bone using a suture or sutures using suture anchors for tendon fixation. In some repair processes, foreign objects, such as suture anchors, staples or screws, are implanted and used to connect tissue to bone. It is also known that manufactured knots are placed in blind holes of a diameter less than that of a knot. In this case, the bone-knot interface is a friction fit and the manufactured knot acts as a suture anchor and these knots are normally covered by tissue after the repair. Suture anchors placed in the bone have the drawbacks including risks of migration, implant breakage and or adverse reactions to the anchor material. Manufactured knots placed in blind holes (not tunnels) eliminate these draw backs but have lower pullout forces than anchors or other implants in all bone types. These manufactured knots are frictionally fit within blind holes and are traditionally used in procedures where the bone is harder than that of the humeral head. A friction fit of the knot is intended to stop the knot from pullout of the same blind hole in which it is inserted and not intended to prevent a suture place in a bone tunnel from cutting though the bone bridge of a transosseous repair.

In the case of partial tears, the superior surface of the rotator cuff is intact. There are two common surgical treatments. One, the partial thickness tear is invasively cut and turned into a compete tear prior to any other surgical steps. Two, implants normally of a diameter near 5mm are twisted or driven through the healthy medial side of partial tear. Thus, there is a need to overcome the invasive nature of partial tissue repairs by a surgical processes. Either by not making a tear larger by cutting it open or by reducing the 5 mm puncture diameter through healthy tissue and reducing tissue trauma caused by twisting of healthy tissue. There is also a need to maintain load beading without the negatives associated with implants such as suture anchors.

Another method of rotator repair is transosseous repair where sutures pass through tissue and bone tunnels having a medial and lateral aperture. Transosseous repair has long been considered a “gold standard” of rotator cuff repair but cyclic biomechanical testing by suture anchor proponents indicate a theoretical/marketing implication that transosseous sutures may cut through bone. Transosseous refers to a complete tunnel though bone having two apertures.

In addition to the invasiveness of presently used surgical methods it is often difficult to pass the flexible sutures though the lumen of a drill guide. Historically, suture used in rotator cuff repair is braided suture thus pushing it forward is as difficult as “pushing a rope.” Therefore, waxes and coatings have been used to facilitate this pushing by making suture more stiff. Although these coatings stiffen suture, it is not always stiff enough to push bone, marrow or other debris in a transosseous exchange. In turn, there is a need to increase reliability.

Once sutures are placed in bone, by any method, suturing soft tissue to the bone is sometimes a second problem. Frequently, surgeons may prefer a stitch that passes more than once through tissue as sutures passed only once though soft rotator cuff tissue are weaker. Often, this is a mattress or other multi pass stitch that requires additional suturing devices and time. This produces a broader surface area and precisely pulls the tissue at two separate points making a stronger repair. This invention overcomes this problem too.

Sutures are commonly joined with knots to complete surgical repairs. Knot usage in rotator cuff repair is sometimes criticized as having the potential to impinge on the under surface of the acromion. Many alterations of knotless suture anchor implants have been developed to address this criticism and are one basis of differentiating knotless suture anchors from suture anchors that require knots. Transosseous rotator cuff repair (placing suture through bone with no anchors) techniques have historically described tying knots in convenient locations relative to a given repair. These convenient locations do not addressed the issue of knot impingement on the undersurface of the acromion or diminish the concerns of critics. Subcortical knot placement addresses this criticism provided the tunnel receiving the knot is of sufficient diameter to allow some back and forth movement during the tying process. The end result is the same anterior-posteror profile of a knotless suture anchor with no impingement

Despite recent statements of equivalence comparing suture anchors and transosseous methods, a marketing based criticism of transosseous rotator cuff repair is “sutures may cut through bone.” Cutting though bone is a failure mode that is debatable and involves complicated biomechanical and outcome research. Sutures can cut soft bone and suture anchors can fail in many ways. The engineering challenge is minimization of these risks. When a suture does cut through bone, it first needs a starting point and increasing the diameter of what must first pull through bone increases the force needed to initiate this potential problem. Subcortcial knot placement greatly increases the diameter of the repair construct at this critical location thus the failure force required.

### SUMMARY OF THE INVENTION

It is an object of the present invention to provide a suture passer for the exchange of suture(s) between sections of a device or delivering suture(s) to a separate device with the purpose of pulling suture(s) through a plane of bone and/or tissue and where benefits of a subcortical knot are realized if used with tunnels of sufficient diameter.

Another object of the present invention is to provide a method to achieve high initial fixations strength, minimize gap formation through subcortial transsoeous knots with multiple tissue passes.

Yet another object of the present invention is to provide a suture passer that is easily loaded with suture material or several preloaded time saving configurations some containing suture(s) and some containing suture(s) combined with loops

The present invention is an improvement in arthroscopic methods and apparatus that use the bone constructs of the patient to attach sutures to torn or dysfunctional tissue. Such surgical methods arthroscopically form a first tunnel in a bone. A second tunnel is arthroscopically made in the same bone and is directed to intersect the first tunnel. In one aspect of such method one of the tunnels is not linear, e.g., is curved as it passes to the intersection of the tunnels.

A suture (or multiple sutures) is passed through one lumen using a tube or other rigid device to contain a single strand or multiple strands of suture or loops of any material that can later be used to pull suture having an enlarged end or other protrusion from the tube, such as a knot or the like which allows the suture to be passed into position. The lumen is passed through the intersecting tunnels one of which may be curved and an end of the suture extends from each of the tunnels. Alternatively, a lead of wire or other material may protrude and be tethered to the suture and or a passing loop that will facilitate the exchange from lumen to lumen.

The suture ends are used to secure the tissue to the bone, such as by arthroscopic tying of the ends, and pulling the tissue against the bone. The suture passer is an elongated member that is in the form a flexible tube or a flexible rod. In a preferred embodiment of the invention the suture passer is an elongated flexible tube containing the suture with an end provided for positioning the sutures. Of course, there may be multiple sutures in the suture passer. These sutures may be of different colors to simplify identification of the ends of each suture to enable tying one end of each suture to the corresponding color of the other end. The suture or sutures may be preloaded into the suture passer to reduce surgical time wherein the multiple sutures are of different colors.

According to the present invention a first material, which is stiffer than the suture material, is joined to the suture material. The first material may be a stiff wire, a braided wire or a monofilament extruded polymer. Whichever first material is chosen it must be sufficiently stiff to easily be inserted through the elongated suture passer. The distal end of the first material may have a U-shape or teardrop-shape. The first material serves to guide the suture material through the elongated tube of the suture passer and provides a means to grasp the suture from the second tunnel using a hook or loop. The suture passer may also be preloaded with sutures combined with a secondary loop of material capable of pulling suture from adjacent bone tunnels to form a basis of creating a mattress stitch or other broad surface stitch without using a secondary instrument aside from the described guide and suture passer tube. Use of this loop moves any suture(s) into multiple tunnels only after the guide itself is completely removed from the patient.

In an alternative embodiment, the suture material is held in place in a longitudinal slot placed along the length of a solid suture passer tube. Such an embodiment provides increased stiffening and load bearing as the suture passer is inserted into the first tunnel. As an alternative to the U-shape and teardrop tethers, a section of shrink tubing of sufficient length can be shrunk over and thereby encasing the suture(s) to aid suture positioning as an assembly that stows suture.

The present invention also uses a bone tunnel of sufficient diameter to facilitate subcortical transosseous knot placement that yields a suture construct repair profile similar to that of a knotless anchor and decreases the likelihood of transosseous sutures cutting trough bone. The lateral bone tunnel of the present method is particularly well suited for subcortical knot placement as it is not typically covered by the rotator cuff in completion of this surgical procedure.

The present invention also provides a method that uses the bone constructs of the patient to attach sutures to torn or dysfunctional tissue. The method employs the specially designed suture passer that passes a suture through an arthroscopic guide formed tunnel in a bone wherein a lateral curved first tunnel intersects a medial second tunnel that in its making has pierced the healthy side of a partial cuff tear. The suture is attached to a first material that is stiffer than the suture material, the first material having a grasping means located at its distal end and placed inside an elongated passer. The elongated passer is inserted through the guide and into the first tunnel and when it reached the intersection with the second tunnel the grasping means is trapped by a hook or loop from a device placed in the second tunnel through the trephine which made the tunnel. The suture is retrieved, then the guide is removed to join and tie the first end of the suture and the second end of the suture over tissue to pull the tissue against the bone. In this case there is a single pass through the cuff know as a simple stitch.

If the guide is used to make two adjacent tunnels, two different preloaded elongated passer designs can be used. One with sutures one with suture and a loop. With the guide removed, one tunnel has two sutures and the adjacent tunnel has one suture and one loop. The loop is used to pull one of the two sutures over the rotator cuff and into adjacent to occupy the space where the loop had been. This makes a multi-pass box stitch similar to a mattress stitch that is stronger than a simple stitch. In the case of a partial tear, the superior surface of the tear is left intact with either the simple stitch or box stitch approach. Multiple variations of this method are possible by altering the number of adjacent tunnels and preloaded configurations. Preloaded configurations may alter the number of sutures or loops. The joining of the suture ends can be tied leaving the knots in arbitrary locations or specifically placed in a subcortical location for added protection for the bone bridge of the transosseous tunnels.

Other objects features and advantages of the present invention will become apparent from the following detailed description of the invention taken in conjunction with the accompanying drawings.

### BRIEF DESCRIPTION OF THE DRAWINGS

Having described the invention in general terms, reference will now be made to the accompanying drawings, which are not necessarily drawn to scale, and wherein:

FIG. 1 illustrates a rotator cuff drill guide in place with a trephine piercing a torn rotator cuff wherein a stand of suture material is shown passing through the drill guide, rotator cuff tissue, into and out of the humeral head, and exiting the central lumen of the trephine;

FIG. 2 illustrates three arthroscopic simple stitches that may be formed when the sutures used in the present invention are in place thereby providing a repaired rotator cuff;

FIG. 3A is a stylus for use in a drill guide having a central lumen for forming a tunnel in bone;

FIG. 3B is a trephine guide pin that fits into a straight drill guide lumen with enough clearance for a trephine;

FIG. 3C is an arthroscopic trephine;

FIG. 3D is an offset hook probe that is capable of grasping the distal end of a first suture material and pulling the first material having a suture joined thereto through a second tunnel;

FIG. 3E is a suture passer of the prior art with suture loosely attached;

FIG. 4 illustrates insertion of an arcuate lumen rotator cuff drill guide leading with the stylus;

FIG. 5 illustrates a trephine guide pin that has been passed through the straight drill guide lumen;

FIG. 6 illustrates a trephine inserted into a tunnel to a optional calibration point on the trephine that advances the tip past the bone void left by the trephine guide pin;

FIG. 7 illustrates the trephine partially retracted to a second calibration mark, a prior art suture stylus with suture being advanced through a tunnel in the bone and an offset hook probe in a position ready to retrieve a suture;

FIG. 8 illustrates the suture lodged in the bone void left by the stylus after being left behind by the suture stylus, and the hook probe/loop or grasping instrument which has been passed through the trephine to capture the suture;

FIG. 9 illustrates two forms of one of the embodiments of the elongated hollow suture passer of the present invention, the lower suture passer with a preloaded suture and the upper suture passer with self loaded sutures;

FIG. 10 illustrates one embodiment of the grasping means of the first suture material of the present invention shown within a suture passer wherein the first material is twisted to form a teardrop shaped loop where this loop may also be preloaded with sutures or additional loops of a suitable material;

FIG. 11 is another embodiment of the grasping means of the first material of the present invention showing parallel ends of a U-shaped rigid material;

FIG. 12 illustrates a first material preloaded into a suture passer member and externally showing a large loop of first material prior to preloading;

FIG. 13 illustrates a self loaded lumen having a small external loop but having a large loop within a suture passer;

FIG. 14 illustrates placing a preloaded suture passer into a drill guide;

FIG. 15 shows a rotator cuff drill guide having a calibrated trephine;

FIG. 16 illustrates a suture pulled by U-shaped or teardrop shaped wire which is compressed in the passing process;

FIG. 17 shows an open simplified view of a shoulder under arthroscopic rotator cuff repair with the drill guide and suture passing elements removed and illustrating the ends of the suture material ready for fixation;

FIG. 18A illustrates the use of two preloaded suture passers, one with a loop and sutures and one with only sutures; FIG. 18B illustrates the suture from the adjacent tunnel being pulled over the rotator cuff and into the first tunnel; and FIG. 18C illustrates tying a suture that is within two adjacent tunnels and covering the rotator cuff between the medial tunnel apertures, and

FIG. 19 illustrates a shrink tube of sufficient length to be shrunk over the leading edge of suture material.

### DETAILED DESCRIPTION OF THE PREFERRED EMBODIMENT

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be through and complete, and will fully convey the scope of the invention to those skilled in the art.

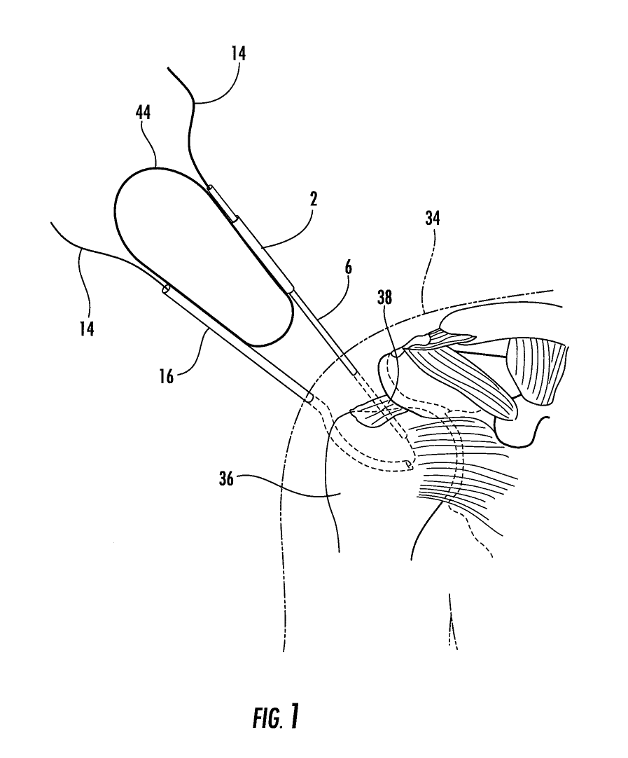

Referring now to the drawings there is shown in FIG. 1 a handle 44 that is used to maintain drill guides 2 and 16 at relative angles for the arthroscopic formation of tunnels. Two arthroscopic portals 30, 32 (shown in FIG. 2) are formed in the shoulder 34, such as by a scalpel. The humeral head 36 and rotator cuff tendons 38 are present. A stand of suture material 14 is shown passing through the drill guide, rotator cuff tissue, into and out of the humeral head, and exiting the central lumen of the trephine 6. The suture material may be multiple strands or loops and of any of the material well-known to those skilled in the art.

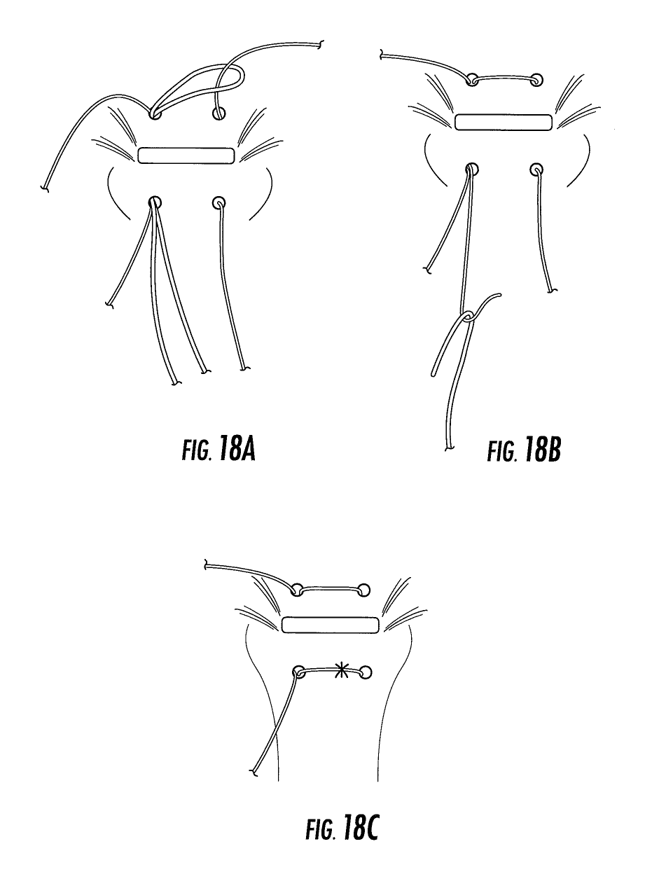

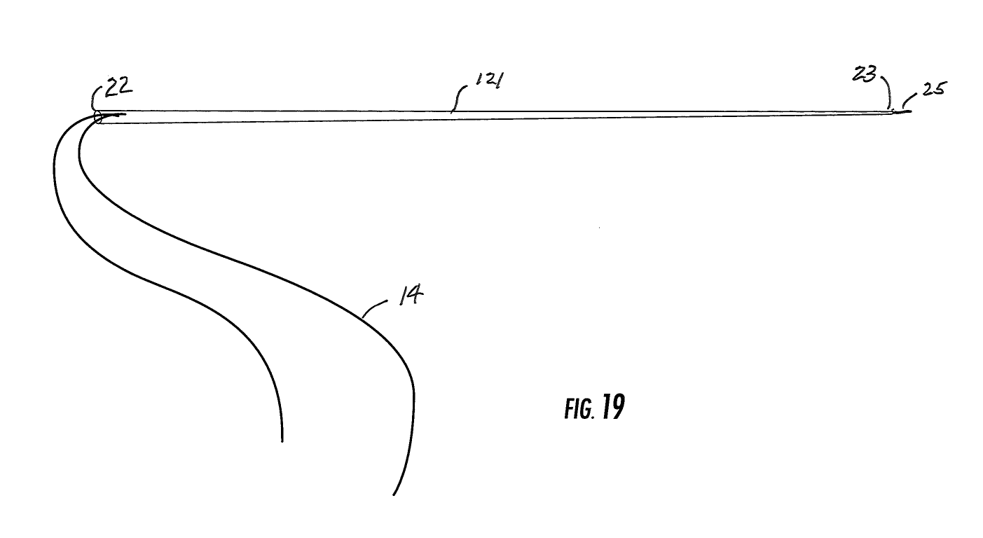

FIG. 17 shows an open simplified view shoulder for rotator cuff repair with the drill guide and suture passing elements removed and the suture 14 ready for fixation. In FIG. 2 there is shown a graphic illustration of three arthroscopic stitches 40 such as might be made with sutures such as those shown in FIG. 17. Alternatively, stitches as shown in FIG. 18C may also be used.

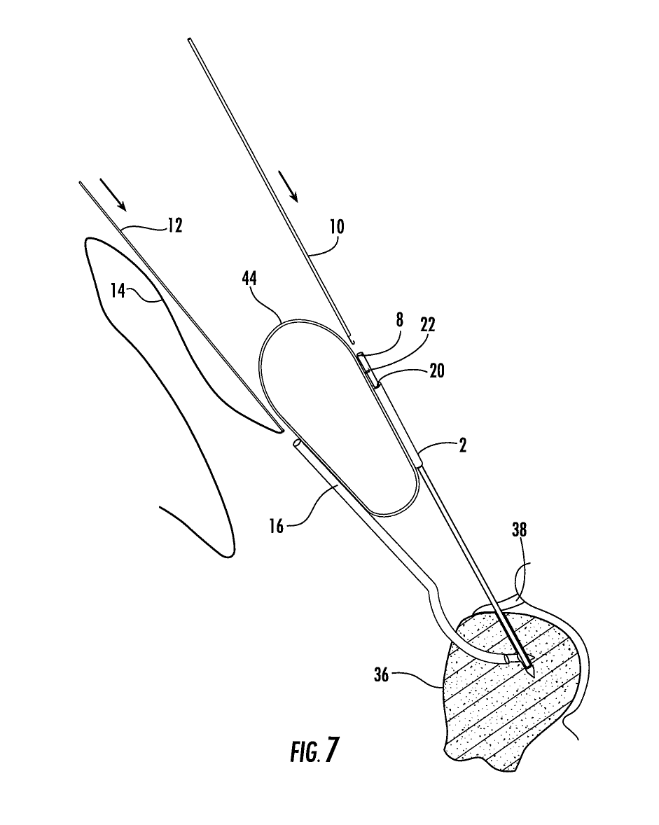

In one of the preferred methods for forming tunnels in the humeral head 36, the embodiment shown in FIG. 4, a curved or arcuate drill guide 16 having a central lumen is inserted into one of the portals. The use of the arcuate drill guide is important in rotator cuff repair to miss neurovascular structures and avoid the acromion. The resulting curved tunnel also transfers biomechanical forces placed on the sutures over a radius of bone to minimize stress points on bone and suture alike. The diameter of the tunnel resulting from this step is sufficient (slightly larger than the knots used) to accomplish subcortial knot placement where the knots are within the lateral tunnel apertures as could be the case in both FIG. 2 and FIG. 18.

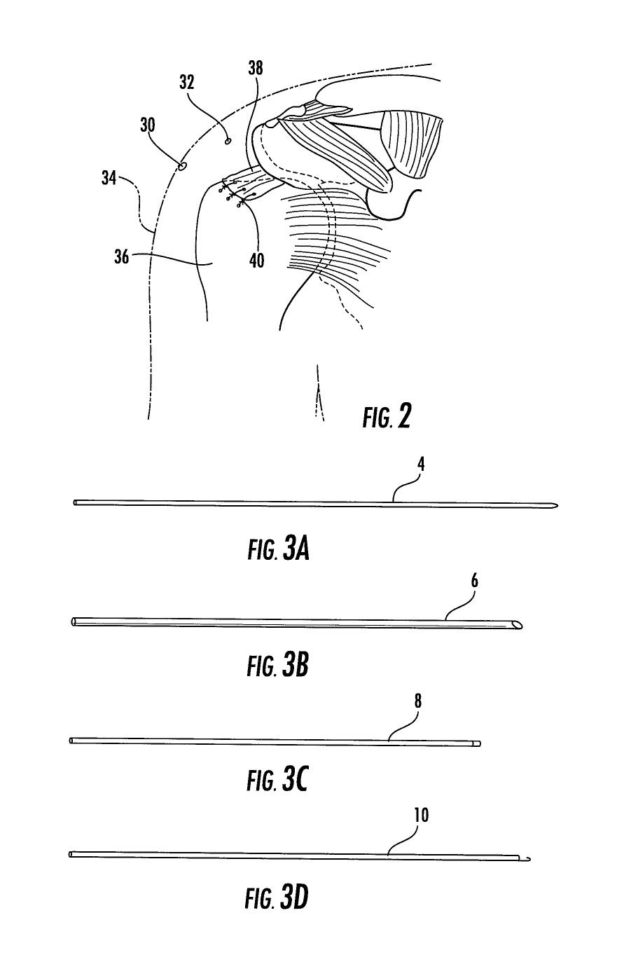

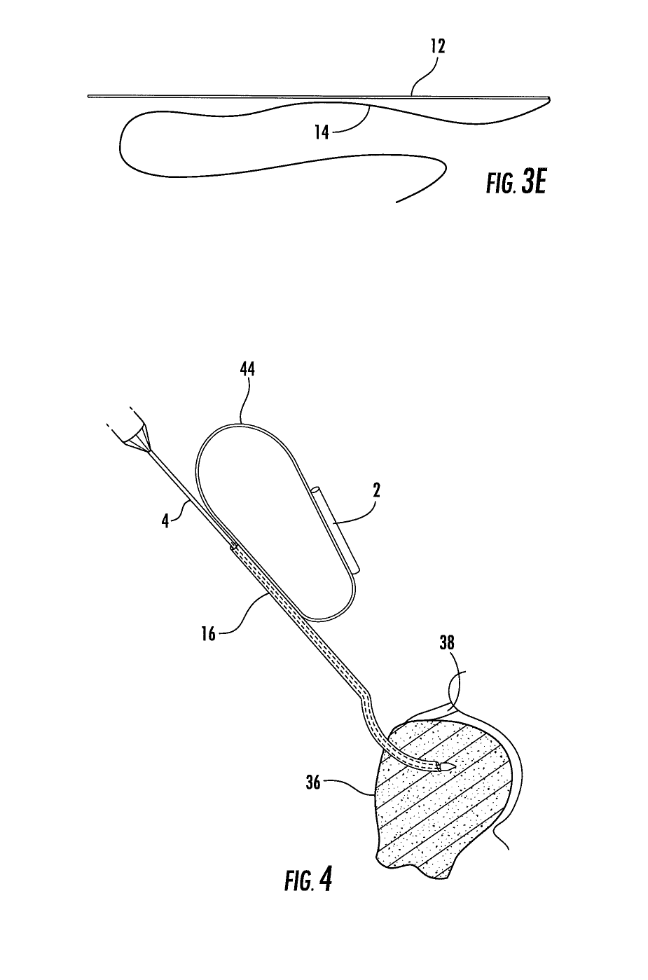

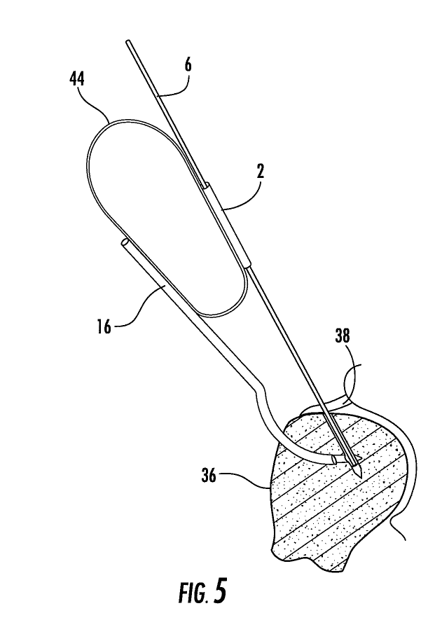

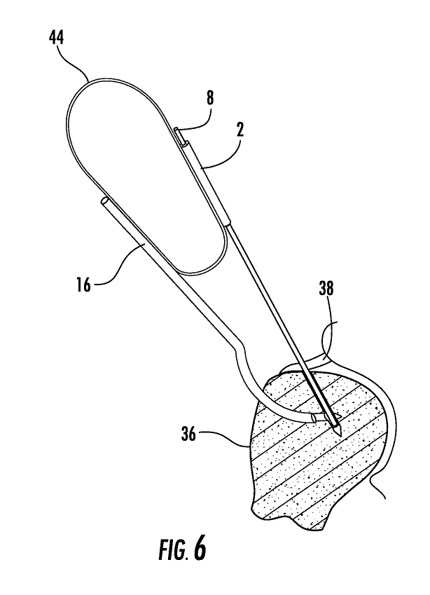

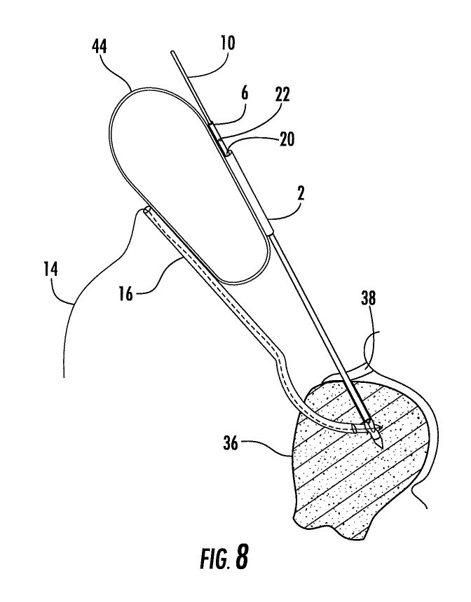

The central lumen of the arcuate drill guide 16 has a protruding flexible stylus 4 therein that is advanced into the humeral head lateral of, or through, the torn rotator cuff. The stylus 4, shown in FIG. 3A, is formed of a memory retaining material, such as nitinol or PEEK. The stylus may have a cutter formed in an end thereof, such as a drill or mill type cutter. Advancement of the arcuate drill guide 16 may be by manual pressure or by assisted manual force using, for example, a mallet, or by a power tool, such as a drill. The arcuate drill guide forms an arcuate tunnel in the bone. After the arcuate drill guide 16 is fully advanced, the stylus is withdrawn, leaving a small void in the bone that is present beyond the leading edge of the arcuate drill guide as shown in FIG. 4 and FIG. 5.

As shown in FIG. 1, the straight drill guide 2 has a lumen therein. A trephine guide pin 6 is positioned within this lumen. The guide pin may be formed of nitinol, stainless steel, PEEK or other materials well-known to those skilled in the art. Sufficient space is present within the drill guide lumen for placement of the trephine guide pin 6 (shown in FIG. 3B), so that the guide pin has a slip fit within the drill guide. Not shown in FIG. 1 is where some rotator cuff tears would allow the curved portion of the guide 16 to also pierce the rotator cuff to achieve two suture fixation points and thus a stronger repair.

This curvilinear action is important as it yields a larger bone bridge (ultimately the source of strength of the described rotator cuff repair) in a limited subacrormial space compared linear intersecting tunnels using the same arthroscopic portal locations. A drill or straight punch would need to be inserted from a more inferior (lower) portal location (where the auxiliary nerve may rest) to yield the same cross sectional area of bone. It also initiates a lateral bone tunnel first (the lateral tunnel is not a resultant a medial tunnel) this allows a greater margin of safety with respect to the auxiliary nerve. A curved lateral tunnel also provides better load distribution than a flat lateral tunnel. This curvilinear delivery is a similar to opening a bottle with an opener but upside down. The diameter of this lateral tunnel is also sufficient to accommodate subcortical knots.

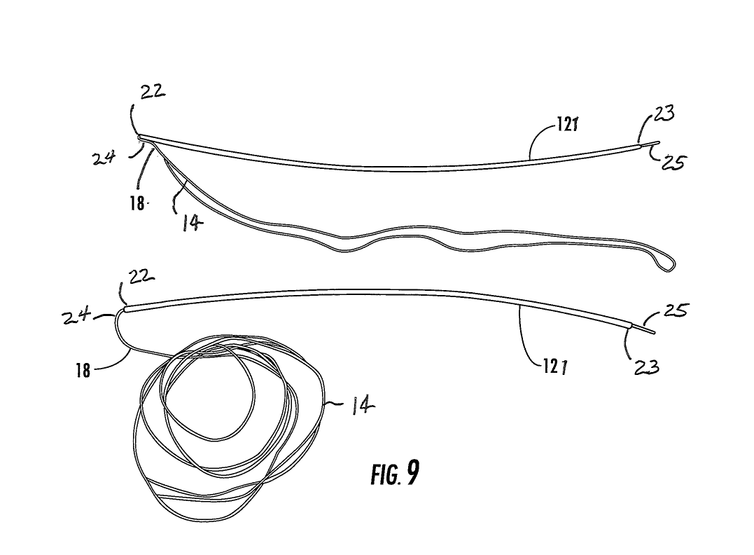

As shown in FIG. 1, the trephine (or cannula) 8, shown in FIG. 3C, is inserted through the lumen of straight drill guide 2. The trephine 8 has a larger diameter than the trephine guide pin 6, but will rotate within the lumen. The trephine 8 enlarges the tunnel, and is moved past the arcuate shaped tunnel formed using the arcuate drill guide 16 as shown most clearly in FIG. 6. For example bone morphogenic proteins or other growth factors may be injected through the lumens. As shown in FIG. 7 and FIG. 15 the trephine may have calibration marks 20, 22 to indicate the depth of insertion and retraction of the trephine. The bone tunnels intersect, as shown.

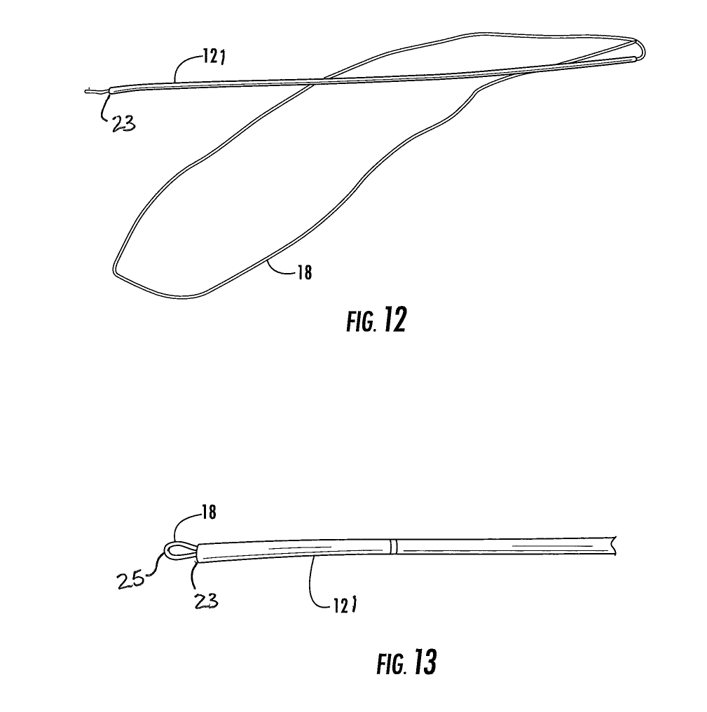

Two embodiments of the suture passer 121 of the present invention are shown in FIG. 9 wherein the lower embodiment shows a elongated hollow suture passer with a preloaded suture and the upper embodiment shows a suture passer with self loaded sutures. The suture passer of the present invention is directed to a device for attaching sutures to torn or dysfunctional tissue for subcortial knot placement. An elongated flexible member 121 capable of insertion into a tunnel in a bone and having a proximal end 22 and a distal end 23 is provided. There is a first material 18 having a proximal end 24 and a distal end 25. The proximal end 24 of the first material 18 is joined to a suture material 14. The distal end 25 of the first material 18 is formed into a means for grasping said distal end by a separate device. The first material 18 is carried by said elongated flexible member 121 into said tunnel.

With the trephine in place, but with the stylus 4 and the trephine guide pin 6 removed from the drill guides, one or more strands of rigid material 18 from one of the suture passers 121 as shown in FIG. 9 are passed through the lumens of the drill guides, likely converging through a re-approximated rotator cuff tear, and through the two intersecting bone tunnels. The suture passer 121 may be preloaded with the sutures 14 guided by the first material 18 or self loaded with sutures when needed. The suture 14 also passes through the humeral head, and exits the central lumen. FIG. 14 shows placing a suture loaded suture passer 121 into drill guide 16, like the drill guides shown in FIG. 3E and FIG. 9.

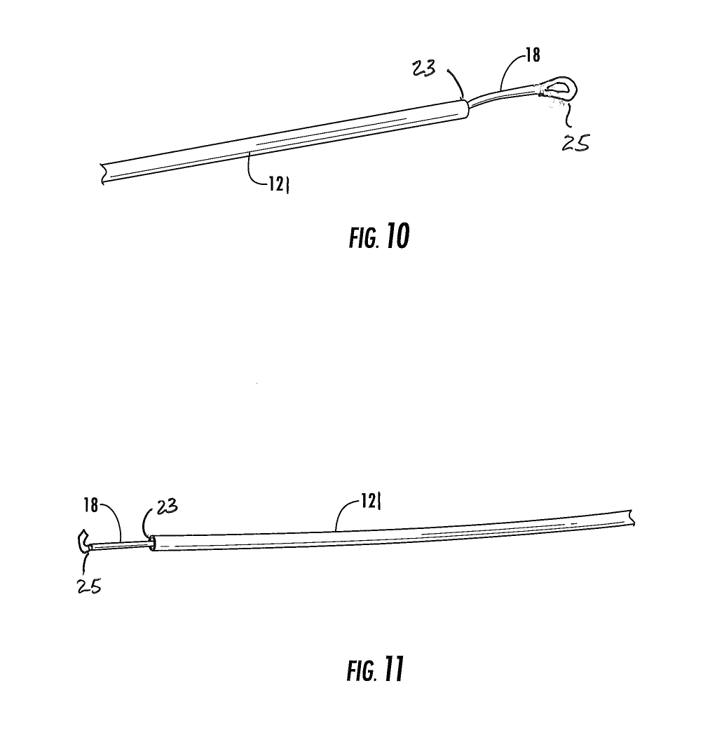

A hook probe (it should be understood that the probe may be in the form of a loop, shown in FIG. 3D) is inserted through the lumen of the trephine to hook the first material 18 advanced by the suture passer 121 at approximately the intersection of the tunnels, as shown in FIG. 8, the suture material having been looped through the teardrop shaped loop 26 shown in FIG. 10. The suture material is advanced past the point of the vacated trephine tunnel.

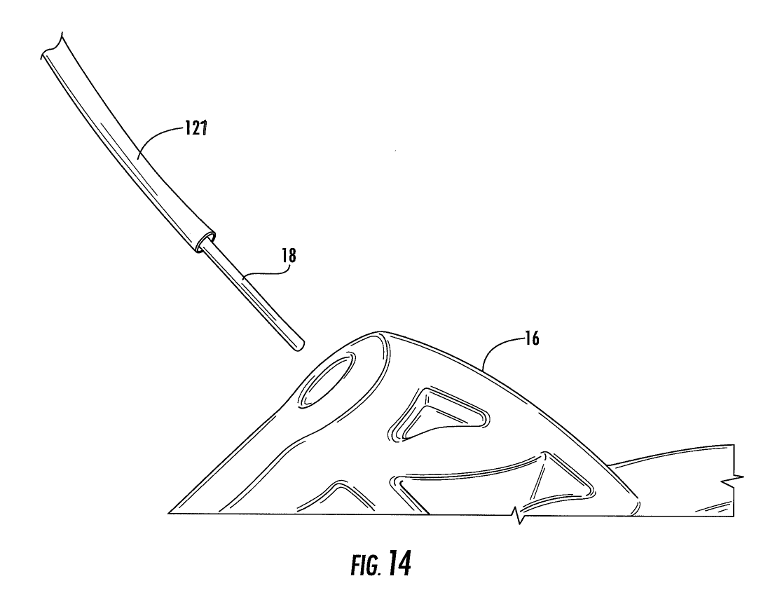

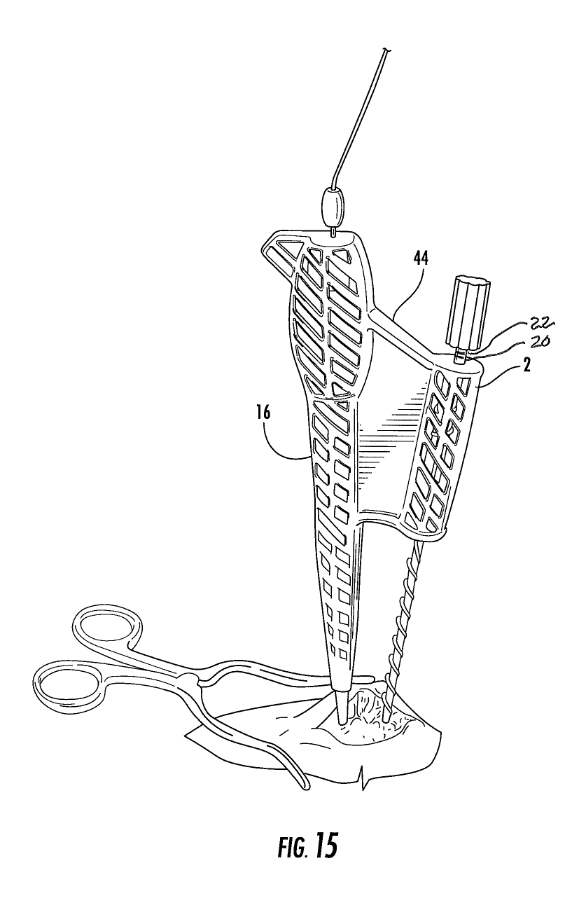

The first material 18, is stiffer that the suture material 14 and may be solid wire, braided wire, coated wire, monofilament extruded polymer. In a preferred embodiment the distal end 25 of the first material 18 is formed into a U-shape with parallel proximal ends as shown in FIG. 11. In FIG. 10 there is shown another embodiment wherein the first material 18 is made of wire having a closed teardrop shape with ends joined by twisting, welding, soldering, gluing, ultrasonic welding or other joining means. In a preloaded configuration the teardrop loop is shorter and tethers the preloaded materials within the elongated member. The teardrop shape is capable of opening much greater in size than the section that is joined. FIG. 11 has the same properties as FIG. 10 but the ends are not twisted or joined. As with FIG. 10 preloaded sutures or additional passing loops are an option or the U alone can be used as a suture aid. The parallel or joined section is captured by a loop shown as in FIG. 12 and FIG. 13, by hook 10 shown in FIG. 3D or probe and subsequently forms its own hook shape to facilitate/enable the passing process.

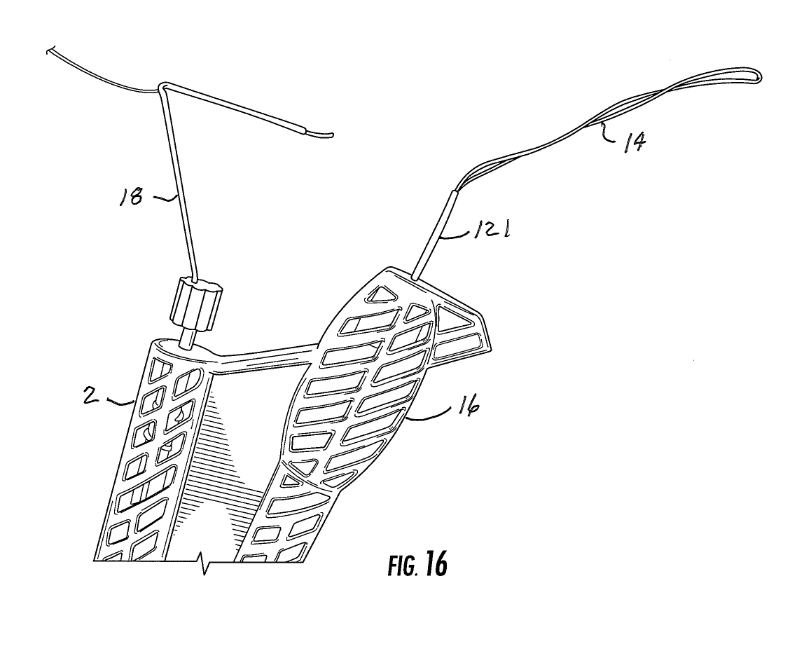

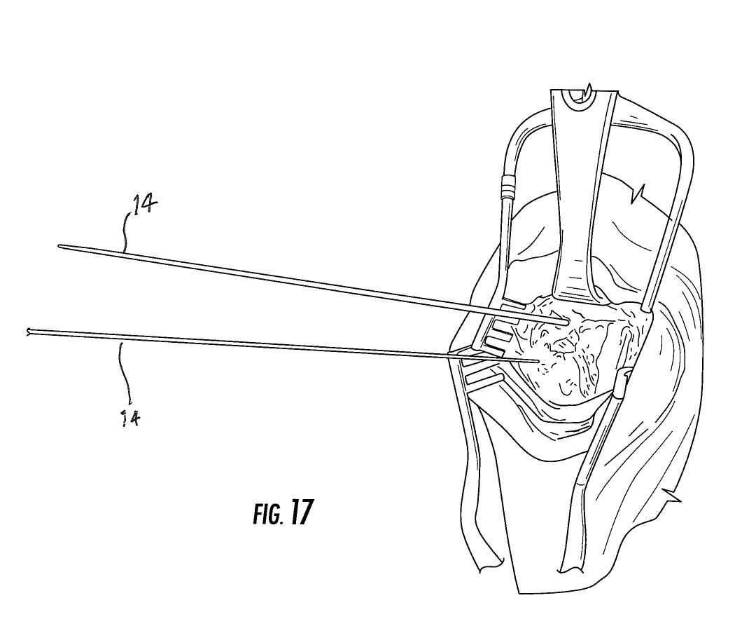

In FIG. 14 a preloaded suture passer 121 having a first material 18 extending from its distal end 23 is shown ready to be passed into drill guide 16. FIG. 15 shows a rotator cuff drill guide 2 having calibrates 20, 22 on the trephine. A shorter trephine with a positive stop obviates a need for a calibration mark. FIG. 16 illustrates a suture 14 in suture passer 121 pulled by U-shaped or teardrop shaped wire 18 through drill guides 2 and 16.

In another embodiment, multiple samples of first material 18 having a wire loop or teardrop at its distal end and joined sutures 14 are placed within the elongated tube 121 that may be slotted or flexible as shown in FIG. 19. The elongated tube 121 provides added rigidity, a bearing surface and suture protection from guide edges and protects bone from being cut by the loop, teardrop or suture during the exchange process. It also creates an option of preloading the suture passer by having suture(s) neatly contained within a tube or a preloaded wire contained within a tube to provide self load options to a surgeon.

The knot location and stitching methods described increase surface area thus load bearing compared to other transosseous methods. A subcortical knot reduces the chance of suture(s) cutting through bone for all types of rotator cuff tears. Additionally, the design of these passers described are particularly well suited for a specific type of rotator cuff tear where the superior surface of the tear is intact but the midsection and or inferior surface of the rotator cuff complex has damage. These tears are known as “partial thickness” tears.

Preloaded tubes designs may also be preloaded with suture combined with a secondary loop (FIG. 18A and FIG. 18B). Being either comprised of suture of the same or different diameter or of a differing material capable of pulling suture from adjacent bone tunnels once the guide used to make the tunnels is removed to form a basis of creating a mattress-like box or other high load bearing stitch without using a secondary instrument aside from the described guide and passing tube. FIGS. 18A, 18B and 18C show a humeral head 36 wherein a suture passer has been used (left tunnel) and the tube and wire loop have already been removed in an arthroscopic procedure. The right tunnel shows only one suture 14 (this tunnel also could contain a secondary loop or a multitude of sutures but this not shown for simplicity.) FIG. 18A shows the secondary loop loaded with the suture 14 from the right tunnel (this would be done externally via a portal). FIG. 18A also illustrates the loop from a first tunnel is used to capture a suture from an adjacent tunnel. FIG. 18B shows the suture 14 from the right tunnel being pulled over the superior surface of the rotator cuff then back through the left tunnel. FIG. 18C shows the suture 14 originally in the right tunnel now in both tunnels and making a mattress/box stitch when joined between the lateral entrance points of the left and right tunnels. The knot location shown in FIG. 18C is arbitrary and is between the lateral tunnel apertures. FIG. 18C infers that this knot and subcortial knots could be placed within the lateral tunnel apertures creating subcortial knot placement to increase repair strength. The joining knot is not shown as a subcortial knot, for clarity. If it were to be shown as subcortial, the knot(s) would rest within the lateral tunnel aperture. Different preloaded versions containing multiple sutures or loops may be used to move sutures through and over the cuff surface. The corresponding guide used with the invention can pierce tissue as it makes a medial tunnel and once the guide is removed from bone the contents of the elongated member are in position to move the suture over tissue and into adjacent tunnels to complete a rotator cuff repair.

Using common sliding/locking knots and equipment familiar to those knowledgeable in arthroscopic knot formation a knot can be placed below the cortex to avoid potential impingement of a knot between the undersurface of the acromion and repair site. The resulting subcortical knot placement provides the same profile as a knotless suture anchors but is implant free. It is also intuitive that a knot resting under the cortex as contrary to a knot resting between adjacent tunnels as shown in FIG. 18A will lesson the likelihood that a suture will pull through bone by greatly increasing surface area.

As an alternative to the described teardrop or U-shaped distal end 25 of the first material 121, a section of shrink tubing of sufficient length can be shrunk over the leading edges of suture FIG. 19. This embodiment resembles that shown in FIG. 12 but with no wire and a smaller shrunken tube itself would be captured pulling the affixed sutures in tow. The suture material may be further contained in a solid elongated member having a slot (longitudinally partial outside the length of the member) providing additional stiffening and load bearing.

Sutures of different colors can be preloaded to simplify identification to enable tying one end of each of said sutures to the corresponding color of the other end. The elongated member 121 may also contain calibration or contain a physical stop to facilitate a more accurate insertion depth for use with a particular complimentary device. The elongated member 121 may also have a sharp distal end for tissue piercing. In another embodiment provides for use of a tube as described above where the proximal (joined or parallel) ends protrude from the tube to facilitate passing. In some embodiments of the present invention the elongated member 21 may be larger at the proximal end 22 than at the distal end 23.

Benefits of the present invention over the use of suture anchors include the introduction of minimal foreign material in the patient, a larger “healing footprint” (which is variable with the distance between lumens) and the use of lumens as injection ports for plate rich/poor blood/growth factors or other growth factors. This method of arthroscopic bone/suture tunnel creation and suture passing also has applications in shoulder laberal repair, Achilles tendon, posterior cruciate ligament and anterior cruciate ligament repair, without, or at least reducing, the requirement of suture anchors, staples or screws. The geometry of the suture passer relates to an arthroscopic creation of bone tunnels and simultaneous suture passing to repair a torn or partially torn rotator cuff.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A suture passer for attaching sutures to torn or dysfunctional tissue for standard knot placement or subcortial knot placement comprising:
(a) an elongated flexible member capable of insertion into a tunnel in a bone said member having a proximal end and a distal end;
(b) a first material having a proximal end and a distal end, said proximal end of said first material being joined to a suture material or this same suture material in combination with a functional open or closed passing loop of a suitable material, said first material being stiffer than said suture material or passing loop;
(c) said distal end of said first material being formed into a means for grasping said distal end by a separate device; and
(d) said first material being carried by said elongated member into said tunnel.

2. The suture passer according to claim 1 wherein said elongated member is a tube sized to contain multiple sutures.

3. The suture passer according to claim 1 wherein said elongated member has a longitudinal slot placed along the length of said member sized to hold sutures in place.

4. The suture passer according to claim 2 wherein said multiple sutures are of different colors to enable tying one end of each of said sutures to the corresponding color of the other end.

5. The suture passer according to claim 1 wherein said first material is a member of the group consisting of solid wire, braided wire, and monofilament extruded polymer.

6. The suture passer according to claim 2 wherein said elongated member has a larger cross-section at the proximal end than the distal end.

7. The suture passer according to claim 1 wherein said elongated member is preloaded with suture material and/or an open or closed passing loop.

8. The suture passer according to claim 7 wherein said elongated member is shrink wrapped.

9. The suture passer according to claim 1 wherein the distal end of said elongated member is slotted.

10. The suture passer according to claim 1 wherein the proximal ends of said suture material extend outside of the proximal end of said elongated member for ease of handling.

11. The suture passer according to claim 1 wherein said means for grasping is in the form of a U-shape.

12. The suture passer according to claim 1 wherein said means for grasping is in the form of a tear drop.

13. The suture passer according to claim 1 wherein said distal end of said elongated member has sharp edge for tissue piercing.

14. A method that uses the bone constructs of the patient to attach sutures to torn or dysfunctional tissue for subcortial knot placement comprising;
(a) arthroscopically forming two tunnels in a bone wherein said first tunnel intersects said second tunnel where the trephine making a medial tunnel may or may not pierce tissue prior to contacting bone;
(b) passing an elongated member carrying a first material joined to a suture material, said suture material having a first end and a second end and said first material having a grasping means at the distal end thereof into one of said tunnels;
(c) grasping the grasping means at the intersection of said tunnels and pulling said first material and suture through said second tunnel;
(d) removing said elongated member from said first tunnel; and
(e) securing said first end of said suture and said second end of said suture over tissue to pull said tissue against said bone.

15. The method according to claim 14 wherein said elongated member carries multiple sutures or open/closed passing loops.

16. The method according to claim 14 wherein said elongated member having said first material also contains multiple sutures combined with an open/closed passing loop(s) that is(are) used to shuttle sutures between tunnels crossing over tissue and between sets of adjacent transosseous tunnels where tissue is pierced during the formation of the medial half of each individual transosseous bone tunnel.

17. The method according to claim 14 wherein said elongated member when removed from said tunnel leaves in situ contents of said elongated member containing suture(s) and suture loop(s) of which the combination has pierced the medial healthy side of partially torn rotator cuff tissue without making the tear complete.

18. The method according to claim 14 wherein the healthy medial side of a partially torn rotator cuff is bridged over by suture crossing between sets of intersecting tunnels when suture from one tunnel is inserted into said loop and pulled into said second tunnel.

19. A method of using a subcortical knots to increase suture surface area at a stress concentration point of transosseous tunnels at or near the lateral tunnel aperture to increase the force required to compromise the integrity of a transosseous bone tunnel wherein the lateral tunnel is not covered by tissue after tying and wherein the lateral tunnel readily accepts a knot or multiple knots stacked in a series.

20. The method according to claim 19 wherein tied sutures rest in one transosseous tunnel.

21. The method according to claim 19 wherein tied sutures rest in multiple transosseous tunnels.

22. A method of using a subcortical knots (knots placed level with, bisecting or below the cortex) thereby reducing the anterior posterior profile of a rotator cuff repair to minimize subacroimial impingement post surgery wherein the lateral tunnel is not covered by tissue after tying and wherein the lateral tunnel readily accepts a knot or multiple knots stacked in a series.

23. The method according to claim 19 wherein tied sutures rest in one transosseous tunnel.

24. The method according to claim 19 wherein tied sutures rest in multiple transosseous tunnels.
